(19) United States
(12) Patent Application Publication
(10) Pub. No.: US 2010/0179355 A1
(43) Pub. Date: Jul. 15, 2010

(54) PROCESSES FOR MAKING DIALKYL ETHERS FROM ALCOHOLS

(75) Inventors: Mark Andrew Harmer, Landenberg, PA (US); Michael B. Damore, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/676,193

(22) PCT Filed: Sep. 5, 2008
(86) PCT No.: PCT/US2008/075309

§ 371 Date: Mar. 3, 2010

Related U.S. Application Data
(60) Provisional application No. 60/970,104, filed on Sep. 5, 2007.

(51) Int. Cl. C07C 41/09

(52) U.S. Cl. 568/698

(57) ABSTRACT

Processes for preparing dibutyl ethers from 2-butanol using an ionic liquid.

This application claims priority from, and the benefit of, U.S. Provisional Application No. 60/970,104, filed Sep. 5, 2007, which is by this reference incorporated in its entirety as a part hereof for all purposes.

### TECHNICAL FIELD

This invention is concerned with processes for preparing dibutyl ethers from 2-butanol.

### BACKGROUND

Ethers such as dibutyl ether are useful as solvents and as diesel fuel cetane enhancers. See, for example, Kotrba, “Ahead of the Curve”, Ethanol Producer Magazine, November 2005; and WO 01/18154, wherein an example of a diesel fuel formulation comprising dibutyl ether is disclosed.

The production of ethers from alcohol, such as the production of dibutyl ether from butanol, is known and is generally described in Kara et al, Kirk-Othmer Encyclopedia of Chemical Technology, Fifth Ed., Vol. 10, Section 5.3, pp. 567˜583. The reaction is generally carried out via the dehydration of an alcohol by sulfuric acid, or by catalytic dehydration over ferric chloride, copper sulfate, silica, or silica-alumina at high temperatures. Bringue et al [J. Catalysis (2006) 244:33-42] disclose thermally stable ion-exchange resins for use as catalysts for the dehydration of 1-pentanol to di-n-pentyl ether. WO 07/38360 discloses a method for making polytrimethylene ether glycols in the presence of an ionic liquid.

A need nevertheless remains for commercially-advantageous processes to prepare ethers from alcohols.

### SUMMARY

The inventions disclosed herein include processes for the preparation of dialkyl ethers such as dibutyl ether from alcohols, the use of such processes, and the products obtained and obtainable by such processes.

Features of certain of the processes of this invention are described herein in the context of one or more specific embodiments that combine various such features together. The scope of the invention is not, however, limited by the description of only certain features within any specific embodiment, and the invention also includes (1) a subcombination of fewer than all of the features of any described embodiment, which subcombination may be characterized by the absence of the features omitted to form the subcombination; (2) each of the features, individually, included within the combination of any described embodiment; and (3) other combinations of features formed by grouping only selected features of two or more described embodiments, optionally together with other features as disclosed elsewhere herein. Some of the specific embodiments of the processes hereof are as follows:

In the processes disclosed herein, a dibutyl ether is prepared in a reaction mixture by (a) contacting 2-butanol with at least one homogeneous acid catalyst in the presence of at least one ionic liquid to form (i) a dibutyl ether phase of the reaction mixture that comprises a dibutyl ether, and (ii) an ionic liquid phase of the reaction mixture; and (b) separating the dibutyl ether phase of the reaction mixture from the ionic liquid phase of the reaction mixture to recover a dibutyl ether product; wherein an ionic liquid is represented by the structure of the following formula:

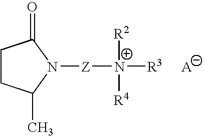

wherein:

in the cation, Z is —(CH₂)ₙ— where n is an integer from 2 to 12; and R², R³ and R⁴ are each independently selected from the group consisting of H, —CH₃, —CH₂CH₃, and C₃ to C₆ straight-chain or branched monovalent alkyl radicals; and

A⁻ is an anion selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂₁CF₂OCF₂CF₂SO₃]⁻, [CF₃CF₂OCF₂CF₂SO₃]⁻, [(CF₂HCF₂SO₂)₂N]⁻, and [(CF₃CFHCF₂SO₂)₂N]⁻.

Ethers, such as the dialkyl ethers produced by the processes hereof, are useful as solvents, plasticizers and as additives in transportation fuels such as gasoline, diesel fuel and jet fuel.

### DETAILED DESCRIPTION

There are herein disclosed processes for preparing dialkyl ethers in the presence of at least one ionic liquid and at least one acid catalyst. Where a homogeneous acid catalyst is used, these processes provide an advantage in that the product dialkyl ether can be recovered in a product phase that is separate from an ionic liquid phase that contains an ionic liquid and an acid catalyst.

In the description of the processes hereof, the following definitional structure is provided for certain terminology as employed in various locations in the specification:

An “alkane” or “alkane compound” is a saturated hydrocarbon having the general formula CₙH₂ₙ₊₂, and may be a straight-chain, branched or cyclic compound.

An “alkene” or “alkene compound” is an unsaturated hydrocarbon that contains one or more carbon-carbon double bonds, and may be a straight-chain, branched or cyclic compound.

An “alkoxy” radical is a straight-chain or branched alkyl group bound via an oxygen atom.

An “alkyl” radical is a univalent group derived from an alkane by removing a hydrogen atom from any carbon atom: —CₙH₂ₙ₊₁ where n=1. The alkyl radical may be a C₁˜C₂₀ straight-chain, branched or cycloalkyl radical. Examples of suitable alkyl radicals include methyl, ethyl, n-propyl, i-propyl, n-butyl, t-butyl, n-pentyl, n-hexyl, cyclohexyl, n-octyl, trimethylpentyl, and cyclooctyl radicals.

An “aromatic” or “aromatic compound” includes benzene and compounds that resemble benzene in chemical behavior.

An “aryl” radical is a univalent group whose free valence is to a carbon atom of an aromatic ring. The aryl moiety may contain one or more aromatic rings and may be substituted by inert groups, i.e. groups whose presence does not interfere with the reaction. Examples of suitable aryl groups include phenyl, methylphenyl, ethylphenyl, n-propylphenyl, n-butylphenyl, t-butylphenyl, biphenyl, naphthyl and ethylnaphthyl radicals.

A “fluoroalkoxy” radical is an alkoxy radical in which at least one hydrogen atom is replaced by a fluorine atom.

A “fluoroalkyl” radical is an alkyl radical in which at least one hydrogen atom is replaced by a fluorine atom.

A “halogen” is a bromine, iodine, chlorine or fluorine atom.

A “heteroalkyl” radical is an alkyl group having one or more heteroatoms.

A “heteroaryl” radical is an aryl group having one or more heteroatoms.

A “heteroatom” is an atom other than carbon in the structure of a radical.

“Optionally substituted with at least one member selected from the group consisting of”, when referring to an alkane, alkene, alkoxy, alkyl, aryl, fluoroalkoxy, fluoroalkyl, heteroalkyl, heteroaryl, perfluoroalkoxy, or perfluoroalkyl radical or moiety, means that one or more hydrogens on a carbon chain of the radical or moiety may be independently substituted with one or more of the members of a recited group of substituents. For example, an optionally substituted —C₂H₅ radical or moiety may, without limitation, be —CF₂CF₃, —CH₂CH₂OH or —CF₂CF₂I where the group of substituents consist of F, I and OH.

A “perfluoroalkoxy” radical is an alkoxy radical in which all hydrogen atoms are replaced by fluorine atoms.

A “perfluoroalkyl” radical is an alkyl radical in which all hydrogen atoms are replaced by fluorine atoms.

In the processes disclosed herein, a dibutyl ether is prepared in a reaction mixture by (a) contacting 2-butanol with at least one homogeneous acid catalyst in the presence of at least one ionic liquid to form (i) a dibutyl ether phase of the reaction mixture that comprises a dibutyl ether, and (ii) an ionic liquid phase of the reaction mixture; and (b) separating the dibutyl ether phase of the reaction mixture from the ionic liquid phase of the reaction mixture to recover a dibutyl ether product; wherein an ionic liquid is represented by the structure of the following formula:

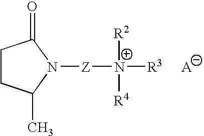

wherein:

in the cation, Z is —(CH₂)ₙ— where n is an integer from 2 to 12; and R², R³ and R⁴ are each independently selected from the group consisting of H, —CH₃, —CH₂CH₃, and C₃ to C₆ straight-chain or branched monovalent alkyl radicals; and

A⁻ is an anion selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂₁CF₂OCF₂CF₂SO₃]⁻, [CF₃CF₂OCF₂CF₂SO₃]⁻, [(CF₂HCF₂SO₂)₂N]⁻, and [(CF₃CFHCF₂SO₂)₂N]⁻.

Ionic liquids are organic compounds that are liquid at room temperature (approximately 25° C.). They differ from most salts in that they have very low melting points, they tend to be liquid over a wide temperature range, and have been shown to have high heat capacities. Ionic liquids have essentially no vapor pressure, and they can either be neutral, acidic or basic. The properties of an ionic liquid will show some variation according to the identity of the cation and anion. However, a cation or anion of an ionic liquid useful for this invention can in principle be any cation or anion such that the cation and anion together form an organic salt that is fluid at or below about 100° C.

The physical and chemical properties of ionic liquids will show some variation according to the identity of the cation and/or anion. For example, increasing the chain length of one or more alkyl chains of the cation will affect properties such as the melting point, hydrophilicity/lipophilicity, density and solvation strength of the ionic liquid. Choice of the anion can affect, for example, the melting point, the water solubility and the acidity and coordination properties of the composition. Effects of choice of cation and anion on the physical and chemical properties of ionic liquids are reviewed by Wasserscheid and Keim [Angew. Chem. Int. Ed. (2000) 39:3772-3789] and Sheldon [Chem. Commun. (2001) 2399-2407].

Ionic liquids suitable for use in a process hereof can be synthesized by the general process of contacting levulinic acid or an ester thereof with a diamine in the presence of a catalyst and hydrogen gas to form an N-hydrocarbyl pyrrolidine-2-one. The pyrrolidine-2-one is then converted to the appropriate ionic liquid by quaternizing the non-ring nitrogen of the pyrrolidine-2-one. These pyrrolidone-based ionic liquids are green ionic liquids that can be prepared from inexpensive renewable biomass feedstock. This type of process is further discussed in U.S. Pat. No. 7,157,588, which is incorporated in its entirety as a part hereof for all purposes.

An ionic liquid may be present in the reaction mixture in an amount of about 0.1% or more, or about 2% or more, and yet in an amount of about 25% or less, or about 20% or less, by weight relative to the weight of the 2-butanol present therein.

A catalyst suitable for use in a process hereof is a substance that increases the rate of approach to equilibrium of the reaction without itself being substantially consumed in the reaction. In preferred embodiments, the catalyst is a homogeneous catalyst in the sense that the catalyst and reactants occur in the same phase, which is uniform, and the catalyst is molecularly dispersed with the reactants in that phase.

In one embodiment, suitable acids for use herein as a homogeneous catalyst are those having a pKa of less than about 4; in another embodiment, suitable acids for use herein as a homogeneous catalyst are those having a pKa of less than about 2.

In one embodiment, a homogeneous acid catalyst suitable for use herein may be selected from the group consisting of inorganic acids, organic sulfonic acids, heteropolyacids, fluoroalkyl sulfonic acids, metal sulfonates, metal trifluoroacetates, compounds thereof and combinations thereof. In yet another embodiment, the homogeneous acid catalyst may be selected from the group consisting of sulfuric acid, fluorosulfonic acid, phosphorous acid, p-toluenesulfonic acid, benzenesulfonic acid, phosphotungstic acid, phosphomolybdic acid, trifluoromethanesulfonic acid, nonafluorobutanesulfonic acid, 1,1,2,2-tetrafluoroethanesulfonic acid, 1,1,2,3,3,3-hexafluoropropanesulfonic acid, bismuth triflate, yttrium triflate, ytterbium triflate, neodymium triflate, lanthanum triflate, scandium triflate, and zirconium triflate.

A catalyst may be present in the reaction mixture in an amount of about 0.1% or more, or about 1% or more, and yet in an amount of about 20% or less, or about 10% or less, or about 5% or less, by weight relative to the weight of the 2-butanol present therein.

The reaction may be carried out at a temperature of from about 50 degrees C. to about 300 degrees C. In one embodiment, the temperature is from about 100 degrees C. to about 250 degrees C. The reaction may be carried out at a pressure of from about atmospheric pressure (about 0.1 MPa) to about 20.7 MPa. In a more specific embodiment, the pressure is from about 0.1 MPa to about 3.45 MPa. The reaction may be carried out under an inert atmosphere, for which inert gases such as nitrogen, argon and helium are suitable.

In one embodiment, the reaction is carried out in the liquid phase. In an alternative embodiment, the reaction is carried out at an elevated temperature and/or pressure such that the product dibutyl ethers are present in a vapor phase. Such vapor phase dibutyl ethers can be condensed to a liquid by reducing the temperature and/or pressure. The reduction in temperature and/or pressure can occur in the reaction vessel itself, or alternatively the vapor phase can be collected in a separate vessel, where the vapor phase is then condensed to a liquid phase.

The time for the reaction will depend on many factors, such as the reactants, reaction conditions and reactor, and may be adjusted to achieve high yields of dibutyl ethers. The reaction can be carried out in batch mode, or in continuous mode.

An advantage to the use of an ionic liquid in this reaction is that, as a result of the formation of the dibutyl ether product, the dibutyl ether product resides in a first phase (a “dibutyl ether phase”) of the reaction mixture that is separate from a second phase (an “ionic liquid phase”) in which the ionic liquid and catalyst reside. Thus the dibutyl ether product or products (in the dibutyl ether phase) is/are easily recoverable from the acid catalyst (in the ionic liquid phase) by, for example, decantation.

In another embodiment, the separated ionic liquid phase may be recycled for addition again to the reaction mixture. The conversion of 2-butanol to one or more dibutyl ethers results in the formation of water. Therefore, where it is desired to recycle the ionic liquid contained in the ionic liquid phase, it may be necessary to treat the ionic liquid phase to remove water. One common treatment method for the removal of water is the use of distillation. Ionic liquids have negligible vapor pressure, and the catalysts useful in this invention generally have boiling points above that of water; therefore it is generally possible when distilling the ionic liquid phase to remove water from the top of a distillation column, whereas an ionic liquid and a catalyst would be removed from the bottom of the column. Methods of distillation applicable to the separation of water from an ionic liquid are further discussed in Section 13, “Distillation” of Perry's Chemical Engineers' Handbook, 7^{th} Ed. (McGraw-Hill, 1997). In further steps, catalyst residue may be separated from an ionic liquid by filtration or centrifugation, or catalyst residue may be returned to the reaction mixture along with the ionic liquid.

The separated and/or recovered dibutyl ether phase can optionally be further purified and can be used as such.

In various other embodiments of this invention, an ionic liquid formed by selecting any of the individual cations described or disclosed herein, and by selecting any of the individual anions described or disclosed herein, may be used in a reaction mixture to prepare a dibutyl ether. Correspondingly, in yet other embodiments, a subgroup of ionic liquids formed by selecting (i) a subgroup of any size of cations, taken from the total group of cations described and disclosed herein in all the various different combinations of the individual members of that total group, and (ii) a subgroup of any size of anions, taken from the total group of anions described and disclosed herein in all the various different combinations of the individual members of that total group, may be used in a reaction mixture to prepare a dibutyl ether. In forming an ionic liquid, or a subgroup of ionic liquids, by making selections as aforesaid, the ionic liquid or subgroup will be used in the absence of the members of the group of cations and/or anions that are omitted from the total group thereof to make the selection, and, if desirable, the selection may thus be made in terms of the members of the total group that are omitted from use rather than the members of the group that are included for use.

Each of the formulae shown herein describes each and all of the separate, individual compounds that can be assembled in that formula by (1) selection from within the prescribed range for one of the variable radicals, substituents or numerical coefficients while all of the other variable radicals, substituents or numerical coefficients are held constant, and (2) performing in turn the same selection from within the prescribed range for each of the other variable radicals, substituents or numerical coefficients with the others being held constant. In addition to a selection made within the prescribed range for any of the variable radicals, substituents or numerical coefficients of only one of the members of the group described by the range, a plurality of compounds may be described by selecting more than one but less than all of the members of the whole group of radicals, substituents or numerical coefficients. When the selection made within the prescribed range for any of the variable radicals, substituents or numerical coefficients is a subgroup containing (i) only one of the members of the whole group described by the range, or (ii) more than one but less than all of the members of the whole group, the selected member(s) are selected by omitting those member(s) of the whole group that are not selected to form the subgroup. The compound, or plurality of compounds, may in such event be characterized by a definition of one or more of the variable radicals, substituents or numerical coefficients that refers to the whole group of the prescribed range for that variable but where the member(s) omitted to form the subgroup are absent from the whole group.

The manner in which advantageous attributes and effects would be obtainable from the processes hereof is described in the form of a series of prophetic examples (Examples 1˜2), as described below. The embodiments of these processes on which the examples are based are representative only, and the selection of those embodiments to illustrate the invention does not indicate that conditions, arrangements, approaches, regimes, reactants, techniques or protocols not described in these examples are not suitable for practicing these processes, or that subject matter not described in these examples is excluded from the scope of the appended claims and equivalents thereof.

### General Materials and Methods

The following abbreviations are used:

Nuclear magnetic resonance is abbreviated NMR; gas chromatography is abbreviated GC; gas chromatography-mass spectrometry is abbreviated GC-MS; thin layer chromatography is abbreviated TLC; thermogravimetric analysis (using a Universal V3.9A TA instrument analyser (TA Instruments, Inc., Newcastle, Del.)) is abbreviated TGA. Centigrade is abbreviated C, mega Pascal is abbreviated MPa, gram is abbreviated g, kilogram is abbreviated Kg, milliliter(s) is abbreviated ml(s), hour is abbreviated hr or h; weight percent is abbreviated wt %; milliequivalents is abbreviated meq; melting point is abbreviated Mp; differential scanning calorimetry is abbreviated DSC.

1-Butyl-2,3-dimethylimidazolium chloride, 1-hexyl-3-methylimidazolium chloride, 1-dodecyl-3-methylimidazolium chloride, 1-hexadecyl-3-methyl imidazolium chloride, 1-octadecyl-3-methylimidazolium chloride, imidazole, tetrahydrofuran, iodopropane, acetonitrile, iodoperfluorohexane, toluene, 2-butanol, oleum (20% SO₃), sodium sulfite (Na₂SO₃, 98%), and acetone were obtained from Acros (Hampton, N.H.). Potassium metabisulfite (K₂S₂O₅, 99%), was obtained from Mallinckrodt Laboratory Chemicals (Phillipsburg, N.J.). Potassium sulfite hydrate (KHSO₃.xH₂O, 95%), sodium bisulfite (NaHSO₃), sodium carbonate, magnesium sulfate, phosphotungstic acid, ethyl ether, 1,1,1,2,2,3,3,4,4,5,5,6,6-tridecafluoro-8-iodooctane, trioctyl phosphine and 1-ethyl-3-methylimidazolium chloride (98%) were obtained from Aldrich (St. Louis, Mo.). Sulfuric acid and methylene chloride were obtained from EMD Chemicals, Inc. (Gibbstown, N.J.). Perfluoro(ethylvinyl ether), perfluoro(methylvinyl ether), hexafluoropropene and tetrafluoroethylene were obtained from DuPont Fluoroproducts (Wilmington, Del.). 1-Butyl-methylimidazolium chloride was obtained from Fluka (Sigma-Aldrich, St. Louis, Mo.). Tetra-n-butylphosphonium bromide and tetradecyl(tri-n-hexyl)phosphonium chloride were obtained from Cytec (Canada Inc., Niagara Falls, Ontario, Canada). 1,1,2,2-Tetrafluoro-2-(pentafluoroethoxy)sulfonate was obtained from SynQuest Laboratories, Inc. (Alachua, Fla.).

### Preparation of Anions

### (A) Synthesis of potassium 1,1,2,2-tetrafluoroethanesulfonate (TFES-K) ([HCF₂CF₂SO₃]⁻)

A 1-gallon Hastelloy® C276 reaction vessel was charged with a solution of

potassium sulfite hydrate (176 g, 1.0 mol), potassium metabisulfite (610 g, 2.8 mol) and deionized water (2000 ml). The pH of this solution was 5.8. The vessel was cooled to 18 degrees C., evacuated to 0.10 MPa, and purged with nitrogen. The evacuate/purge cycle was repeated two more times. To the vessel was then added tetrafluoroethylene (TFE, 66 g), and it was heated to 100 degrees C. at which time the inside pressure was 1.14 MPa.

The reaction temperature was increased to 125 degrees C. and kept there for 3 hr. As the TFE pressure decreased due to the reaction, more TFE was added in small aliquots (20-30 g each) to maintain operating pressure roughly between 1.14 and 1.48 MPa. Once 500 g (5.0 mol) of TFE had been fed after the initial 66 g precharge, the vessel was vented and cooled to 25 degrees C. The pH of the clear light yellow reaction solution was 10-11. This solution was buffered to pH 7 through the addition of potassium metabisulfite (16 g).

The water was removed in vacuo on a rotary evaporator to produce a wet solid. The solid was then placed in a freeze dryer (Virtis Freezemobile 35×1; Gardiner, N.Y.) for 72 hr to reduce the water content to approximately 1.5 wt % (1387 g crude material). The theoretical mass of total solids was 1351 g. The mass balance was very close to ideal and the isolated solid had slightly higher mass due to moisture. This added freeze drying step had the advantage of producing a free-flowing white powder whereas treatment in a vacuum oven resulted in a soapy solid cake that was very difficult to remove and had to be chipped and broken out of the flask. The crude TFES-K can be further purified and isolated by extraction with reagent grade acetone, filtration, and drying.

¹⁹F NMR (D₂O) δ −122.0 (dt, J_{FH}=6 Hz, J_{FF}=6 Hz, 2F); −136.1 (dt, J_{FH}=53 Hz, 2F).

¹H NMR (D₂O) δ 6.4 (tt, J_{FH}=53 Hz, J_{FH}=6 Hz, 1H).

% Water by Karl-Fisher titration: 580 ppm.

Analytical calculation for C₂HO₃F₄SK: C, 10.9; H, 0.5; N, 0.0. Experimental results: C, 11.1; H, 0.7; N, 0.2.

Mp (DSC): 242 degrees C.

TGA (air): 10% wt. loss @ 367 degrees C., 50% wt. loss @ 375 degrees C.

TGA (N₂): 10% wt. loss @ 363 degrees C., 50% wt. loss @ 375 degrees C.

### (B) Synthesis of potassium-1,1,2-trifluoro-2-(perfluoroethoxy)ethanesulfonate (TPES-K)

A 1-gallon Hastelloy® C276 reaction vessel was charged with a solution of

potassium sulfite hydrate (88 g, 0.56 mol), potassium metabisulfite (340 g, 1.53 mol) and deionized water (2000 ml). The vessel was cooled to 7 degrees C., evacuated to 0.05 MPa, and purged with nitrogen. The evacuate/purge cycle was repeated two more times. To the vessel was then added perfluoro(ethylvinyl ether) (PEVE, 600 g, 2.78 mol), and it was heated to 125 degrees C. at which time the inside pressure was 2.31 MPa. The reaction temperature was maintained at 125 degrees C. for 10 hr. The pressure dropped to 0.26 MPa at which point the vessel was vented and cooled to 25 degrees C. The crude reaction product was a white crystalline precipitate with a colorless aqueous layer (pH=7) above it.

The ¹⁹F NMR spectrum of the white solid showed pure desired product, while the spectrum of the aqueous layer showed a small but detectable amount of a fluorinated impurity. The desired isomer is less soluble in water so it precipitated in isomerically pure form. The product slurry was suction filtered through a fritted glass funnel, and the wet cake was dried in a vacuum oven (60 degrees C., 0.01 MPa) for 48 hr. The product was obtained as off-white crystals (904 g, 97% yield).

¹⁹F NMR (D₂O) δ −86.5 (s, 3F); −89.2, −91.3 (subsplit ABq, J_{FF}=147 Hz, 2F);

−119.3, −121.2 (subsplit ABq, J_{FF}=258 Hz, 2F);

### −144.3 (dm, J_{FH}=53 Hz, 1F).

¹H NMR (D₂O) δ 6.7 (dm, J_{FH}=53 Hz, 1H).

Mp (DSC) 263 degrees C.

Analytical calculation for C₄HO₄F₈SK: C, 14.3; H, 0.3 Experimental results; C, 14.1; H, 0.3.

TGA (air): 10% wt. loss @ 359 degrees C., 50% wt. loss @ 367 degrees C.

TGA (N₂): 10% wt. loss @ 362 degrees C., 50% wt. loss @ 374 degrees C.

### (C) Synthesis of potassium-1,1,2-trifluoro-2-(trifluoromethoxy)ethanesulfonate (TIES-K)

A 1-gallon Hastelloy® C276 reaction vessel was charged with a solution of

potassium sulfite hydrate (114 g, 0.72 mol), potassium metabisulfite (440 g, 1.98 mol) and deionized water (2000 ml). The pH of this solution was 5.8. The vessel was cooled to −35 degrees C., evacuated to 0.08 MPa, and purged with nitrogen. The evacuate/purge cycle was repeated two more times. To the vessel was then added perfluoro(methylvinyl ether) (PMVE, 600 g, 3.61 mol) and it was heated to 125 degrees C. at which time the inside pressure was 3.29 MPa. The reaction temperature was maintained at 125 degrees C. for 6 hr. The pressure dropped to 0.27 MPa at which point the vessel was vented and cooled to 25 degrees C. Once cooled, a white crystalline precipitate of the desired product formed leaving a colorless clear aqueous solution above it (pH=7).

The ¹⁹F NMR spectrum of the white solid showed pure desired product, while the spectrum of the aqueous layer showed a small but detectable amount of a fluorinated impurity. The solution was suction filtered through a fritted glass funnel for 6 hr to remove most of the water. The wet cake was then dried in a vacuum oven at 0.01 MPa and 50 degrees C. for 48 hr. This gave 854 g (83% yield) of a white powder. The final product was isomerically pure (by ¹⁹F and ¹H NMR) since the undesired isomer remained in the water during filtration.

¹⁹F NMR (D₂O) δ −59.9 (d, J_{FH}=4 Hz, 3F); −119.6, −120.2 (subsplit ABq, J=260 Hz, 2F); −144.9

### (dm, J_{FH}=53 Hz, 1F).

¹H NMR (D₂O) δ 6.6 (dm, J_{FH}=53 Hz, 1H).

% Water by Karl-Fisher titration: 71 ppm.

Analytical calculation for C₃HF₆SO₄K: C, 12.6; H, 0.4; N, 0.0. Experimental results: C, 12.6; H, 0.0; N, 0.1.

Mp (DSC) 257 degrees C.

TGA (air): 10% wt. loss @ 343 degrees C., 50% wt. loss @ 358 degrees C.

TGA (N₂): 10% wt. loss @ 341 degrees C., 50% wt. loss @ 357 degrees C.

### (D) Synthesis of sodium 1,1,2,3,3,3-hexafluoropropanesulfonate (HFPS—Na)

A 1-gallon Hastelloy® C reaction vessel was charged with a solution of

anhydrous sodium sulfite (25 g, 0.20 mol), sodium bisulfite 73 g, (0.70 mol) and of deionized water (400 ml). The pH of this solution was 5.7. The vessel was cooled to 4 degrees C., evacuated to 0.08 MPa, and then charged with hexafluoropropene (HFP, 120 g, 0.8 mol, 0.43 MPa). The vessel was heated with agitation to 120 degrees C. and kept there for 3 hr. The pressure rose to a maximum of 1.83 MPa and then dropped down to 0.27 MPa within 30 minutes. At the end, the vessel was cooled and the remaining HFP was vented, and the reactor was purged with nitrogen. The final solution had a pH of 7.3.

The water was removed in vacuo on a rotary evaporator to produce a wet solid. The solid was then placed in a vacuum oven (0.02 MPa, 140 degrees C., 48 hr) to produce 219 g of white solid which contained approximately 1 wt % water. The theoretical mass of total solids was 217 g. The crude HFPS—Na can be further purified and isolated by extraction with reagent grade acetone, filtration, and drying.

¹⁹F NMR (D₂O) δ −74.5 (m, 3F); −113.1, −120.4

### (ABq, J=264 Hz, 2F); −211.6 (dm, 1F).

¹H NMR (D₂O) δ 5.8 (dm, J_{FH}=43 Hz, 1H).

Mp (DSC) 126 degrees C.

TGA (air): 10% wt. loss @ 326 degrees C., 50% wt. loss @ 446 degrees C.

TGA (N₂): 10% wt. loss @ 322 degrees C., 50% wt. loss @ 449 degrees C.

### Preparation of Ionic Liquids

### (E) Preparation of the Bis-Trifluoromethanesulfonimide Salt of 1-(2-N,N,N-Dimethylpropylaminoethyl)-5-Methyl-Pyrrolidine-2-one

### a) Preparation of 1-(2-N,N-Dimethylaminoethyl)-5-Methyl-Pyrrolidine-2-one

Ethyl levulinate (18.5 g), N,N-dimethylethylenediamine (11.3 g), and 5% Pd/C (ESCAT-142, 1.0 g) were mixed in a 400 ml shaker tube reactor. The reaction was carried out at 150 degrees C. for 8 hr under 6.9 MPa of H₂.

The reactants and products were analyzed by gas chromatography on a HP-6890 GC (Agilent Technologies; Palo Alto, Calif.) and HP-5972A GC-MS detector equipped with a 25M×0.25 mM ID CP-Wax 58 (FFAP) column. The GC yields were obtained by adding methoxyethyl ether as the internal standard. The ethyl levulinate conversion was 99.7%, and the product selectivity for 1-(2-N,N-dimethylaminoethyl)-5-methyl-pyrrolidine-2-one was 98.6%.

### b) Preparation of the Iodide Salt of 1-(2-N,N,N-Dimethylpropylaminoethyl)-5-Methyl-Pyrrolidine-2-one

For the quaternization reaction, purified 1-(2-N,N-dimethylaminoethyl)-5-methyl-pyrrolidine-2-one (1.7 g) was placed in 5 g of dry acetonitrile, and 1.69 g of 1-iodopropane was added. This mixture was refluxed overnight under a nitrogen atmosphere; the reaction was shown to be complete via TLC, yielding the iodide salt of the quaternary ammonium compound. The acetonitrile was then removed under vacuum.

### c) Preparation of the Bis-Trifluoromethanesulfonimide Salt of 1-(2-N,N,N-Dimethylpropylaminoethyl)-5-Methyl-Pyrrolidine-2-one by Anion Exchange

For the anion exchange reaction, the iodide salt (1 g) produced in the quaternization reaction of step (b) was added to water (5 g), and then ethanol (5 g) was added. A stoichiometric amount of bis-trifluoromethanesulfonimide was added and the mixture was stirred for about 24 hours under nitrogen. A separate layer formed at the bottom, orange-red in color, which was quickly washed with water; the upper layer was decanted. The orange-red liquid was then placed in an oven at 100 degrees C. under vacuum for 48 hours to obtain the ionic liquid (bis-trifluoromethanesulfonimide salt of 1-(2-N,N,N-dimethylpropylaminoethyl)-5-methyl-pyrrolidine-2-one). The stability of the ionic liquid was investigated by thermogravimetric analysis as follows: the ionic liquid (79 mg) was heated at 10 degrees C. per minute up to 800 degrees C. using a Universal V3.9A TA instrument analyser (TA Instruments, Inc., Newcastle, Del.); the results demonstrated that the ionic liquid is stable to decomposition up to about 300 degrees C.

### (F) Preparation of the Hexafluorophosphate Salt of 1-(2-N,N,N-Dimethylpropylaminoethyl)-5-Methyl-Pyrrolidine-2-one

### a) Preparation of the Bromide Salt of 1-(2-N,N,N-Dimethylpropylaminoethyl)-5-Methyl-Pyrrolidine-2-one

For the quaternization reaction purified 1-(2-N,N-dimethylaminoethyl)-5-methyl-pyrrolidine-2-one (1 g) synthesized in Example 1 was placed in 5 g of dry acetonitrile, and 0.71 g of 1-bromopropane was added. This mixture was refluxed overnight under a nitrogen atmosphere; the reaction was shown to be complete via TLC, yielding the bromide salt of the quaternary ammonium compound. The acetonitrile was then removed under vacuum.

### b) Preparation of the Hexafluorophosphate Salt of 1-(2-N,N,N-Dimethylpropylaminoethyl)-5-Methyl-Pyrrolidine-2-one by Anion Exchange

For the anion exchange reaction, the bromide salt (0.5 g) produced in the quaternization reaction of Example 2(a) was added to water (5 g), and then ethanol (5 g) was added. A stoichiometric amount of bis-hexafluorophosphate (Sigma-Aldrich) was added, followed by an additional 2 ml of water, and the mixture was stirred for about 24 hours under nitrogen. A separate layer formed at the bottom, which was quickly washed with water; the upper layer was decanted. The remaining liquid was then placed in an oven at 100 degrees C. under vacuum for 48 hours to obtain the ionic liquid; 0.6 g of the ionic liquid was obtained.

### (G) Preparation of the Bromide Salt of 1-(2-N,N,N-Dimethylpentylaminoethyl)-5-Methyl-Pyrrolidine-2-one

### a) Preparation of the Bromide Salt of 1-(2-N,N,N-Dimethylpentylaminoethyl)-5-Methyl-Pyrrolidine-2-one

For the quaternization reaction purified 1-(2-N,N-dimethylaminoethyl)-5-methyl-pyrrolidine-2-one (1 g) synthesized in Example 1(a) was placed in 5 g of dry acetonitrile, and 1.51 g of 1-bromopentane was added. This mixture was refluxed overnight under a nitrogen atmosphere; the reaction was shown to be complete via TLC, yielding the bromide salt of the quaternary ammonium compound. The acetonitrile was then removed under vacuum, yielding the ionic liquid.

### b) Preparation of the Trifluoromethylsulfonate Salt of 1-(2-N,N,N-Dimethylpentylaminoethyl)-5-Methyl-Pyrrolidine-2-one

For the quaternization reaction, purified 1-(2-N,N-dimethylaminoethyl)-5-methyl-pyrrolidine-2-one (13.5 g) from step (a) was placed in 20 g of dry acetonitrile, and 10 g of 1-bromopropane was added. The mixture was heated at 60 degrees C. for 4 hours. Potassium triflate was then added in acetonitrile (9.5 g in 30 ml of acetonitrile). The mixture was stirred for 4 hours at 60 degrees C. and then left overnight at room temperature. The potassium bromide precipitated. The mixture was filtered and the potassium bromide-free solid was placed under vacuum to remove the solvent. The mixture was dried to give the trifluoromethanesulfonate as the anion of the ionic liquid. The product was confirmed by NMR. The final yield of the ionic liquid (trifluoromethylsulfonate salt of 1-(2-N,N,N-dimethylpentylaminoethyl)-5-methyl-pyrrolidine-2-one) was 13 g.

### Example 1

### Conversion of 2-Butanol to Dibutyl Ether

2-Butanol (30 g), 1-(2-N,N,N-dimethylpropylaminoethyl)-5-methyl-pyrrolidine-2-one 1,1,2,2-tetrafluoroethanesulfonate (5 g), and 1,1,2,2-tetrafluoroethanesulfonic acid (0.6 g) are placed in a 200 ml shaker tube. The tube is heated under pressure with shaking for 6 h at 180° C. The vessel is then cooled to room temperature, and the pressure is released. Prior to heating the components are present as a single liquid phase, however the liquid becomes a 2-phase system after reacting and cooling the components. The top phase is expected to contain predominantly dibutyl ether with less than 10% 2-butanol. The bottom phase is expected to contain 1,1,2,2-tetrafluoroethanesulfonic acid, 1-(2-N,N,N-dimethylpropylaminoethyl)-5-methyl-pyrrolidine-2-one 1,1,2,2-tetrafluoroethanesulfonate, and water. The conversion of 2-butanol is expected to be about 90%, as measured by NMR. It is expected that the two liquid phases are very distinct and separate within several minutes (<5 min).

### Example 2

### Conversion of 2-Butanol to Dibutyl Ether

2-Butanol (60 g), 1-(2-N,N,N-dimethylpropylaminoethyl)-5-methyl-pyrrolidine-2-one 1,1,2,2-tetrafluoroethanesulfonate (10 g), and 1,1,2,2-tetrafluoroethanesulfonic acid (1.0 g) are placed in a 200 ml shaker tube. The tube is heated under pressure with shaking for 6 h at 180° C. Prior to heating the components are present as a single liquid phase. After reacting and cooling the components, the liquid becomes a 2-phase system. The top phase is expected to contain greater than 75% dibutyl ether with less than 25% 2-butanol, and does not contain measurable quantities of ionic liquid or catalyst. The bottom phase is shown to contain 1,1,2,2-tetrafluoroethanesulfonic acid, 1-(2-N,N,N-dimethylpropylaminoethyl)-5-methyl-pyrrolidine-2-one 1,1,2,2-tetrafluoroethanesulfonate, water and about 10% by weight 2-butanol relative to the combined weight of the ionic liquid, acid catalyst, water and 2-butanol. The conversion of 2-butanol is estimated to be about 90%. It is expected that the two liquid phases are very distinct and separate within several minutes (<5 min).

What is claimed is:

1. A process for preparing a dibutyl ether in a reaction mixture comprising (a) contacting 2-butanol with at least one homogeneous acid catalyst in the presence of at least one ionic liquid to form (i) a dibutyl ether phase of the reaction mixture that comprises a dibutyl ether, and (ii) an ionic liquid phase of the reaction mixture; and (b) separating the dibutyl ether phase of the reaction mixture from the ionic liquid phase of the reaction mixture to recover a dibutyl ether product; wherein an ionic liquid is represented by the structure of the following formula: 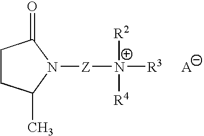
wherein:
in the cation, Z is —(CH₂)ₙ— where n is an integer from 2 to 12; and R², R³ and R⁴ are each independently selected from the group consisting of H, —CH₃, —CH₂CH₃, and C₃ to C₆ straight-chain or branched monovalent alkyl radicals; and
A⁻ is an anion selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, [(CF₂HCF₂SO₂)₂N]⁻, and [(CF₃CFHCF₂SO₂)₂N]⁻.

2. The process of claim 1, wherein a homogeneous acid catalyst is a homogeneous acid catalyst having a pKa of less than about 4.

3. The process of claim 1, wherein the reaction mixture comprises an ionic liquid in an amount of about 0.1% or more, and yet in an amount of about 25% or less, by weight relative to the weight of the 2-butanol present therein.

4. The process of claim 1, wherein a homogeneous acid catalyst is a homogeneous acid catalyst selected from the group consisting of inorganic acids, organic sulfonic acids, heteropolyacids, fluoroalkyl sulfonic acids, metal sulfonates, metal trifluoroacetates, compounds thereof and combinations thereof.

5. The process of claim 1, wherein a homogeneous acid catalyst is a homogeneous acid catalyst selected from the group consisting of sulfuric acid, fluorosulfonic acid, phosphorous acid, p-toluenesulfonic acid, benzenesulfonic acid, phosphotungstic acid, phosphomolybdic acid, trifluoromethanesulfonic acid, nonafluorobutanesulfonic acid, 1,1,2,2-tetrafluoroethanesulfonic acid, 1,1,2,3,3,3-hexafluoropropanesulfonic acid, bismuth triflate, yttrium triflate, ytterbium triflate, neodymium triflate, lanthanum triflate, scandium triflate, and zirconium triflate.

6. The process of claim 1, wherein the reaction mixture comprises a catalyst in an amount of about 0.1% or more, and yet in an amount of about 20% or less, by weight relative to the weight of the 2-butanol present therein.

7. The process of claim 1, which is carried out under an inert atmosphere.

8. The process of claim 1, wherein the dibutyl ether product is in the vapor phase.

9. The process of claim 1, wherein the ionic liquid phase comprises catalyst residue.

10. The process of claim 1, wherein the separated ionic liquid phase is recycled to the reaction mixture.

11. The process of claim 1, wherein water is removed from the separated ionic liquid phase.

12. The process of claim 1, wherein forming the reaction mixture occurs at a temperature of from about 50 degrees C. to about 300 degrees C. and a pressure of from about 0.1 MPa to about 20.7 MPa.

13. The process of claim 1, wherein forming the reaction mixture occurs at a temperature of from about 50 degrees C. to about 300 degrees C. and a pressure of from about 0.1 MPa to about 20.7 MPa, and an ionic liquid is 1-(2-N,N,N-dimethylpropylaminoethyl)-5-methyl-pyrrolidine-2-one 1,1,2,2-tetrafluoroethanesulfonate.

14. The process of claim 1, wherein forming the reaction mixture occurs at a temperature of from about 50 degrees C. to about 300 degrees C. and a pressure of from about 0.1 MPa to about 20.7 MPa, wherein an ionic liquid is 1-(2-N,N,N-dimethylpropylaminoethyl)-5-methyl-pyrrolidine-2-one 1,1,2,2-tetrafluoroethanesulfonate, and a homogeneous acid catalyst is tetrafluoroethanesulfonic acid.
